(19) United States
(12) Patent Application Publication
(10) Pub. No.: US 2010/0202931 A1
(43) Pub. Date: Aug. 12, 2010

(54) Ozone generator apparatus and method for purification of air or liquids

(76) Inventor: Charles E. C. Harris, Chiang Rai (TH)

Correspondence Address: Bartels Law Group, P.O. Box 1999, Burlingame, CA, 94011-1999, US

(21) Appl. No.: 12/634,652

(22) Filed: Dec. 9, 2009

Related U.S. Application Data
(60) Provisional application No. 61/121,123, filed on Dec. 9, 2008.

(51) Int. Cl. B01J 19/12; A61L 2/20; A61L 9/015

(52) U.S. Cl. 422/119; 422/186.3

(57) ABSTRACT

This invention relates to an ozone generator for making ozone using a UV lamp. The ozone generator includes separate first and second sealed containers preferably mounted on a common base. An air pump positioned within the first sealed container draws air into the first sealed container through an air filter. The air pump also causes this filtered air to exit the first sealed container and enter the second sealed container. A UV light source is positioned inside the second sealed container in a manner so as to cause the air from said first sealed container to be radiated by said UV light source as the air flows across the UV light source and towards an output opening in the second sealed container, to cause at least some of the air to be converted into ozone gas prior to the air exiting the second container. A cooling fan and a ballast circuit for the UV light source are preferably also contained within the first sealed container.

### PRIORITY

The present application claims priority to U.S. Provisional Application No. 61/121,123, filed Dec. 9, 2008, entitled Ozone Generator Apparatus and Method for Purification of Air or Liquids, the entirety of which is hereby incorporated by reference.

### FIELD OF THE INVENTION

This invention relates in general to a purification apparatus, and more specifically to an ozone generator having a sealed chamber in which air being drawn into the generator is filtered before being used to generate ozone.

### BACKGROUND

Ozone (O₃) is a gas with strong oxidation properties that has been used for domestic water purification in Europe since the late 1904. Ozone is produced by high-intensity ultraviolet (UV) light or by a high-voltage electric field. The usual byproduct of ozone is oxygen (O₂). Ozone is created when either UV or a corona discharge (CD) is applied to oxygen. The oxygen disassociates into single oxygen atoms which recombine into ozone. Ozone has a half life of about 2 to 12 hours in air and about 20 minutes when dissolved in water. Ozone is 13 times more soluble in water than oxygen. Ozone oxidizes and disinfects and deodorizes. Ozone also microfloculates iron and manganese, and kills bacteria 3,000 times faster than chlorine, kills viruses, algae spores, and some parasites, precipitates heavy metals, controls formation of scales, and oxidizes oils. Ozone also has a fresh smell like that of an electrical storm.

A problem with prior art ozone generators is that air is used to keep the interior of the generator cool. It is this same air that is drawn into the ozone generator and used to create the ozone. Using such a system to filter a pool or a spa results in the introduction of algae and bacteria and many other airborne contaminants and has been found to require daily brushing to remove algae from the pool surfaces and various chemicals to control contamination.

Another problem with prior art ozone generators is that a fan is typically used to circulate outside air into the box. Some ozone generators simply blow air into the generator and other styles filter the air first or afterwards. The problem with filtering air used by a fan for cooling is that the air filter that is used gets clogged quickly because it is not only cleaning the air that the air pumps will consume, but additional millions of liters of cooling air that is being blown into a typical ozone generator box every month. If no air filter is used, the fan blows in air and dust which eventually blankets the components with dirt and an insulating heat retaining film on the same components one is trying to keep cool.

One exemplary prior art ozone generator is taught in U.S. Pat. No. 5,190,648, to Ramsauer. The ozone generator 53 and air pump 54 shown in FIG. 2 of that patent, reproduced as prior art FIG. 1 in this application, are contained in a common cabinet. The air pump 54 used in the Ramsauer generator is a twin bellows type including a pair of bellows 55 and 56 operated by a drive motor 57 that is electrically connected to power leads 58 which also connect to a ballast 59 for the ozone generator 53. The bellows 55 and 56 are connected to tubing 55′ and 56′ which are connected to a T 61 for causing the air pumped by air pump 54 to be fed into one end of a cavity 62 inside the housing 63 of the ozone generator 53. An ultraviolet lamp 64 is mounted in the cavity 62. The airstream that has been radiated by the ultraviolet lamp 64, and thus which contains ozone, flows out from the end of the housing 63 opposite the T 61 into a tube 65, which is in turn is connected to a water tank where the ozone is to be delivered.

As taught in the Ramsauer patent, the ozone generator does not separate the UV lamp housing from the air pump and ballast. In the Ramsauer design, a leak in the ozone chamber would destroy the air pump bellows and potentially other components. The Ramsauer generator also does not filter the air coming into the air pump. Nor does the Ramsauer generator treat the air coming into the air pump in any other way or muffle the noise of the air pump, which makes a considerable racket.

### SUMMARY OF THE INVENTION

The ozone generator according to the present invention provides a low cost, low capacity ozone generator and air pump delivery system that reduces algae and bacteria and other organic matter in a pool or spa, or water storage tank used for home or industrial water or for a drinking water bottle factory, to thereby provide a chemical-free, salt-free, water treatment system, while, at the same time, operating at a much reduced noise level than prior art ozone generators.

Broadly stated, the present invention is an ozone generator that includes a first sealed container having a first opening for enabling air to enter said first sealed container and a second opening for enabling air to exit said first sealed container; an air filter positioned at said first opening of said first sealed container for filtering air entering said container; an air pump positioned inside said first sealed container for drawing air into said container through said air filter and for causing said air to exit through said second opening; a second sealed container having first and second openings, said first opening operatively connected to said second opening of said first sealed container for receiving into said sealed second container air from said first sealed container, said second opening for enabling air to exit said second sealed container; and a UV light source positioned inside said second sealed container in a manner so as to cause the air from said first opening in said second sealed container to be radiated by said UV light source as the air flows towards said second opening of said second sealed container, to cause at least some of the air to be converted into ozone gas.

According to the present invention, ozonated air that is output by the ozone generator is almost sterile and relatively free of organic matter. When this air is coupled to a pool, spa or water storage tank, the fact that it is almost sterile and free of organic materials helps a great deal to keep the water clean and clear. To produce this clean and sterilized air, the ozone generator first filters a small fraction of air by drawing the air into the ozone generator through a filter positioned over a small hole. An air pump contained within a sealed housing in the ozone generator is used to draw the air through the filter. By enclosing the air pump and filter in a sealed space, two benefits are obtained. First, the sealed space acts like a muffler to reduce noise generated by the air pump, and second, the result is clean air within the sealed space that is then fed by the air pump to one or more UV lamps preferably housed in a second sealed container. The ozone is created from the air in this second sealed container and fed to an output tube for coupling to the water tank, pool or spa. The added heat treatment generated first in the sealed housing containing the air pump and second in the second chamber housing the UV lamp just contributes that much more to the elimination of germs and algae and other airborne contaminants.

For the filter media, a conventional filter media that filters out airborne contaminants larger than a predetermined size is used. An exemplary filter media usable with the present invention filters particulate matter of greater than 10 microns in size. A highly efficient HEPA filter media may be used. Another preferable material is a highly efficient filter media typically found used with industrial breathing masks, which filter organic and some chemicals. Because of the low volume of air that is filtered by the ozone generator according to the present invention (only what the air pump draws in), the pore size of the filter media can be very small, smaller than most particles in the air. If one used a cooling fan to cool the whole unit, a HEPA filter would be clogged in a very short time and many of the internal components would overheat and burn up.

Depending on the environment in which the ozone generator is operating, since it does not use a cooling fan, the air drawn into the first sealed container containing the air pump (muffler) becomes heated once it is inside. The air temperature inside the container may reach 50° C. or higher. If the air does get this hot, it acts as a further air treatment to break down organic material, bacteria, or other pathogens in the air. The first chamber also reduces relative humidity which further inhibits germ growth and reduces corrosion on internal components. The air in the sealed container is pumped by the air pump into the second sealed container where the UV lamp further heats the air, such that the temperature may reach 60° to 80° C. to further cause the air to be sterilized. Although this higher temperature reduces the amount of ozone generated by the UV lamp, the trade off is that the air output by the ozone generator is more sterilized as a result.

In a second embodiment of the ozone generator according to the present invention, the sealed container contains two air pumps and the output of each is fed to separate UV lamps in a second housing. This embodiment doubles the output of the ozone generator.

According to an alternate embodiment, the present invention is an ozone generator includes a first sealed container having a first opening for enabling air to enter said first sealed container and a plurality of output openings for enabling air to exit said first sealed container; an air filter positioned at said first opening of said first sealed container for filtering air entering said container; a plurality of air pumps positioned inside said first sealed container for drawing air into said container through said air filter, each air pump output coupled to a separate said output opening for causing said air to exit through said output openings; a second sealed container having a plurality of input openings and one second opening, said input openings operatively connected to corresponding said output opening of said first sealed container for receiving into said sealed second container air from said first sealed container, each input opening coupled to a separate cavity in said second container, said second opening for enabling air to exit said second sealed container; and a plurality of UV light sources each positioned inside a respective cavity in said second sealed container in a manner so as to cause the air from said input openings in said second sealed container to be radiated by said UV light sources as the air flows through to said second opening of said second sealed container, to cause at least some of the air to be converted into ozone gas.

The present invention is an apparatus and method for making ozone with a UV lamp and using an air pump to pump the ozone into water. The generator can also be used in air filtering applications, where the ozone is pumped into the air. In a hotel room that has a cigarette smell or where there has been smoke damage from a fire, for example, the ozone would act to neutralize the smell.

### BRIEF DESCRIPTION OF THE DRAWINGS

Some of the features, advantages, and benefits of the present invention will become more apparent for the following description taken in conjunction with the accompanying drawings, which are presented solely for exemplary purposes and not with the intent to limit the invention thereto, and in which:

FIG. 1 is schematic side elevational view, partly in section, of a prior art ozone generator;

FIG. 2 is a perspective view of an ozone generator according to the present invention;

FIGS. 3-5 show front, side and bottom views of the sealed container/muffler of an ozone generator according to the present invention;

FIG. 6 is a perspective view showing the components of a first embodiment of the ozone generator according to the present invention, with the housing shown in phantom;

FIG. 7 is a bottom view of the ozone generator shown in FIG. 6;

FIG. 8 is a perspective view showing components of a second embodiment of the ozone generator according to the present invention, with the housing shown in phantom;

FIG. 9 is a bottom view of the ozone generator shown in FIG. 8;

FIG. 10 shows a more detailed view of the UV lamp source in the second sealed container of the ozone generator shown in FIG. 6, with the container (housing) shown in phantom;

FIG. 11 shows a more detailed view of the UV lamp source in the second sealed container of the ozone generator shown in FIG. 8, with the container (housing) shown in phantom;

FIG. 12 is a detailed view of a UV lamp connected according to the present invention;

FIG. 13 is a more detailed view of one end of the UV lamp shown in FIG. 12;

FIG. 14 shows a preferred translucent cap for said second container; and

FIG. 15 is a table which shows that, for a typical air pump, the volume of air flow depends upon the depth of the outlet port for the ozone as it enters the water being treated.

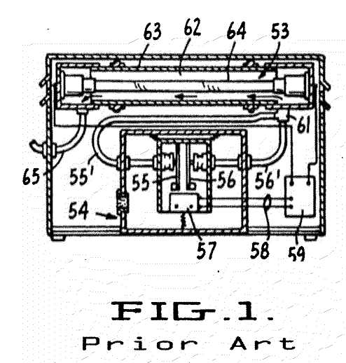

### DETAILED DESCRIPTION OF THE INVENTION

What has been realized by the inventor is that a key factor in the success of both pool and spa filtering is the way the ozone generator makes ozone. When the ozone generator according to the present invention is used, both the pool and spa waters tested negative for Total Coliform and E-coli bacteria and the water became very clean and clear. A local university said the pool passed its “drinking water” standard and the spa almost passed on all points except for 5 points over the scale for the “total dissolved solids” parameter. That parameter may be higher than the others because the spa is just a cement container without any lining and other factors and the source water from a local mountain stream was not resin treated first.

Similarly, the inventor has realized that water storage tanks used in drinking water factories or home drinking water supplies can be kept cleaner as well, avoiding the necessity of chlorine for bottled water or drinking water.

A key feature of the ozone generator according to the present invention is the way the ozone is made. In short, airborne bacteria, algae spores, various organic contaminants, etc., are efficiently filtered out of the air that is used to generate the ozone as the air is drawn into a sealed container by an air pump contained within the container. The sealed container also acts as a muffler to significantly reduce the noise created by the air pump. In an alternate embodiment, the air in the ozone generator is also heated, to thereby dehydrate and cook the air to further sterilize the ozone stream that is sent to the pool and spa. The only air that is allowed into the generator is the small amount of air drawn in by the air pump inside the generator. In one preferred embodiment, featuring a single UV lamp and single air pump, approximately 3 liters of air are sucked into the generator per minute. The generator is a sealed system. Heat comes primarily from the air pump and a UV lamp ballast that also is positioned inside the muffler. Cooling air is not brought inside. Instead, a little muffin fan (which may use a small transformer which also generates some heat) swirls the air inside the sealed container and doubles not only as a protective box, but also as a heat exchanger. The effect is cheap sterile and clean air. This prevents algae and other impurities from being pumped into the pool and spa or water storage tank for drinking water. For example, one exemplary prior art ozone generator required weekly brushing of the pool that the generator was connected to, to remove algae from the pool surfaces. The ozone generator according to the present invention enables the pool to go for a month or two before brushing of pool surfaces is required. A further benefit of limiting the air in the sealed container to well filtered air is that it keeps the components of the ozone generator cleaner and more protected from moisture, which enables these components to last longer.

One embodiment of the present invention incorporates an air filter and a cooling fan to blow filtered air into the ozone generating chamber to keep it cool. Heat reduces the production of ozone, and so there is an advantage to keeping the air cool as it is converted to ozone. However, even without such cooling, the ozone generator according to the present invention make enough ozone to be practical. In another embodiment, the ozone generator first heats and treats the feed air to remove airborne spores and bacteria and then cools the air before it goes into the ozone generating chamber. In one embodiment, a pressure line is inserted between the air pump and ballast chamber and the lamp housing and functions to super heat the air and then the line expands before it enters the ozone chamber to improve ozone production by providing cooler air.

The fact that the ozone generator is a sealed chamber enables the ozone generator to operate very quietly compared to prior art ozone generators. In other words, a user is enabled to enjoy a conversation with the ozone generator just a few feet away. This would be impossible with prior art ozone generators (at least those that do not use a venturi to mix the ozone into the water), which typically have a powerful and noisy air pump installed inside the unit.

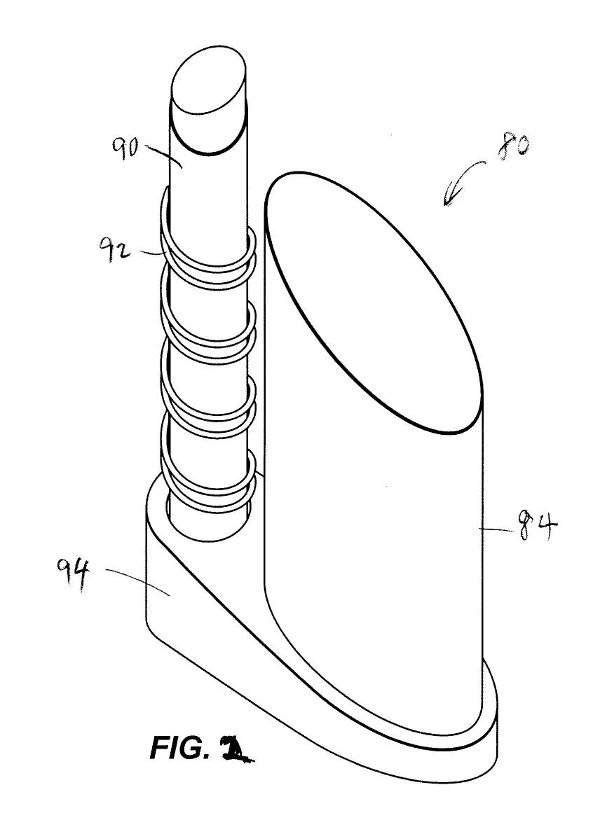

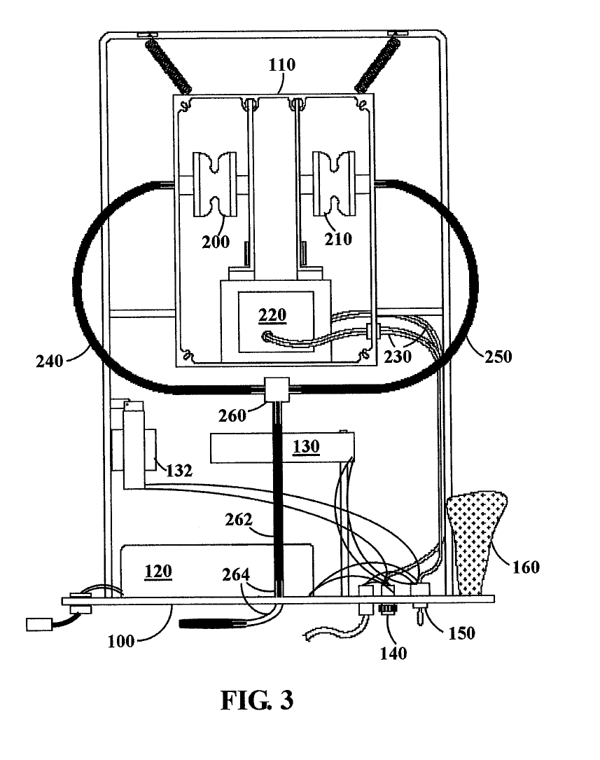

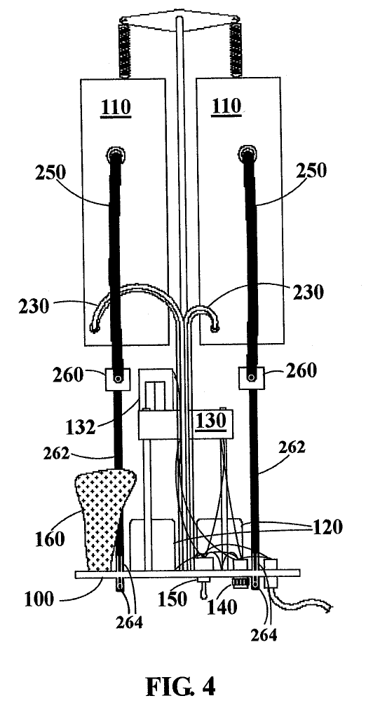

As seen in FIG. 2, the basic ozone generator 80 according to the present invention preferably includes two parts, a sealed container in an elliptical shape, shown at 84, and a separate sealed UV lamp housing, shown at 90. One or more steel rods 92 are wound around lamp housing 90 are preferably used to provide some cooling of the housing and a protective barrier if the lamp housing is touched. Other cooling mechanisms known in the art can also be used. The sealed container 84 is mounted on a plate (as best seen in FIGS. 3-5 below) that is covered by a cover 94.

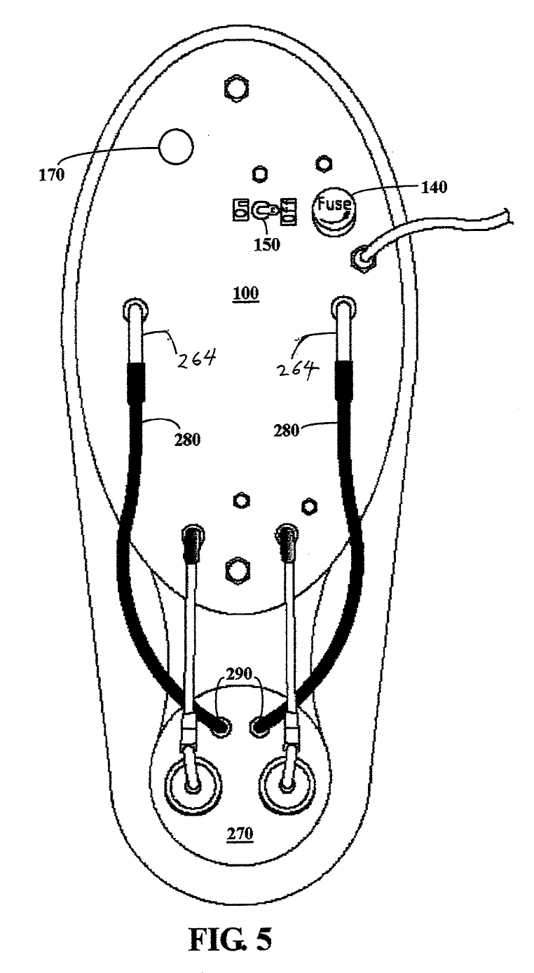

As seen in FIGS. 3-5, which comprise a front view, side view and bottom view of the contents of the sealed container with its elliptical case removed. Mounted on the base 100 of the sealed container is at least one air pump 110, ballast 120, cooling fan 130, fuse 140, on/off switch 150, and air filter 160. The air input hole through which air is drawn into the sealed chamber and through filter 160 is shown at 170 in FIG. 5. The embodiment shown in these figures is of a double ozone generator that contains two of each of these components other than the input filter and the cooling fan.

As best seen in FIG. 3, each air pump 110 is a twin bellows type including a pair of bellows 200 and 210 operated by a drive motor 220 that is electrically connected to power leads 230 which also connect to ballast 120. The bellows 200 and 210 are mounted using springs to dampen the vibrations generated by the bellows. The outputs of bellows 200 and 210 are connected to tubing 240 and 250 which are connected to a T 260 for causing the air pumped by air pump 110 to be coupled out of the sealed container via a tube 264 formed in base 100 for coupling to the UV lamp housing, the base of which is shown in FIG. 5 at 270. Essentially, the air pump sends the air out of its two bellows and to T 260 where the air lines are joined into one and fed to a Neoprene tube 262. This tube attaches to a stainless steel tube 264 that is welded to the base plate 100, the “harp plate” and comes out the bottom end, where another Neoprene tube, as seen at 280 in FIG. 5, attaches to a stainless steel nipple 290 at the bottom 270 of the stainless steel lamp housing. For the double, there are two sets of the above identified components.

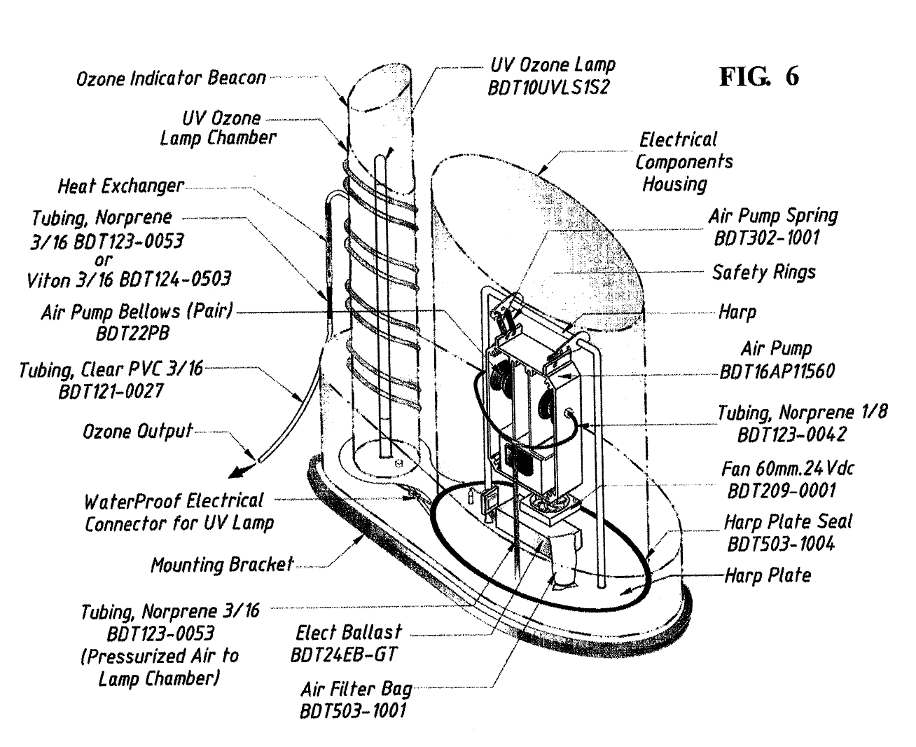

FIG. 6 is a perspective view showing the components of a first embodiment of the ozone generator according to the present invention.

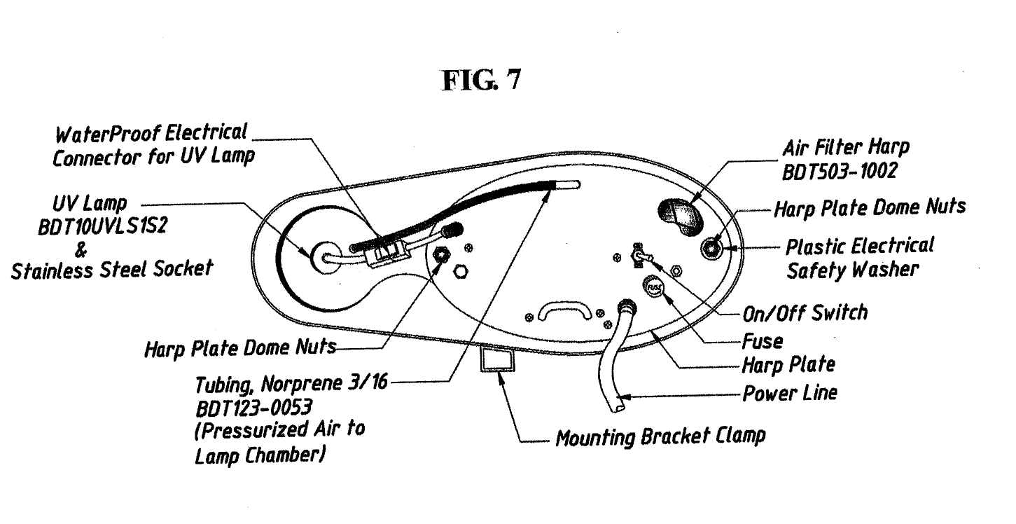

FIG. 7 is a bottom view of the ozone generator shown in FIG. 6.

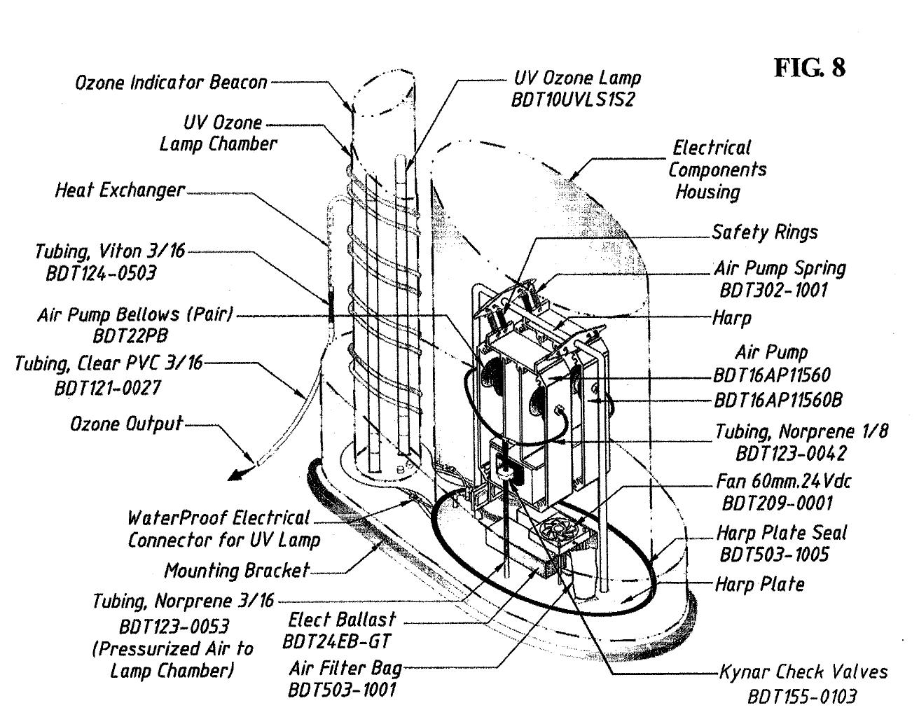

FIG. 8 is a perspective view showing components of a second embodiment of the ozone generator according to the present invention.

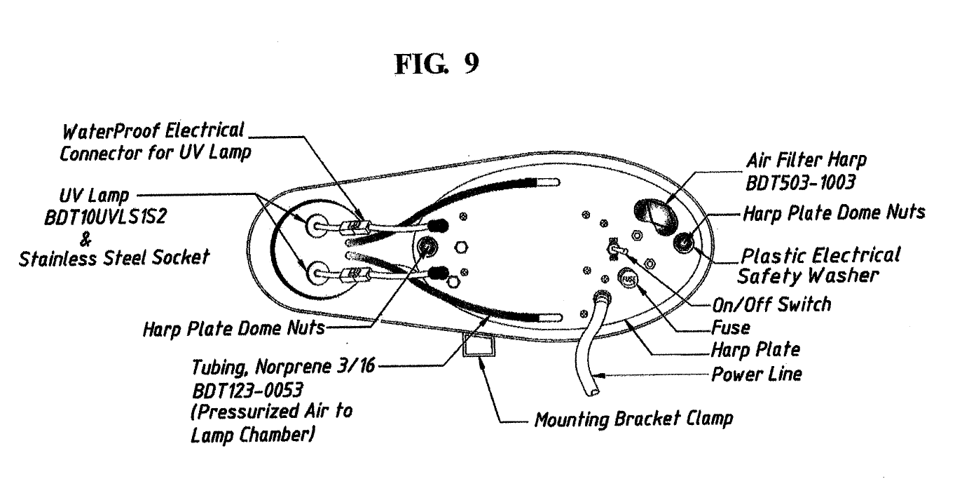

FIG. 9 is a bottom view of the ozone generator shown in FIG. 8.

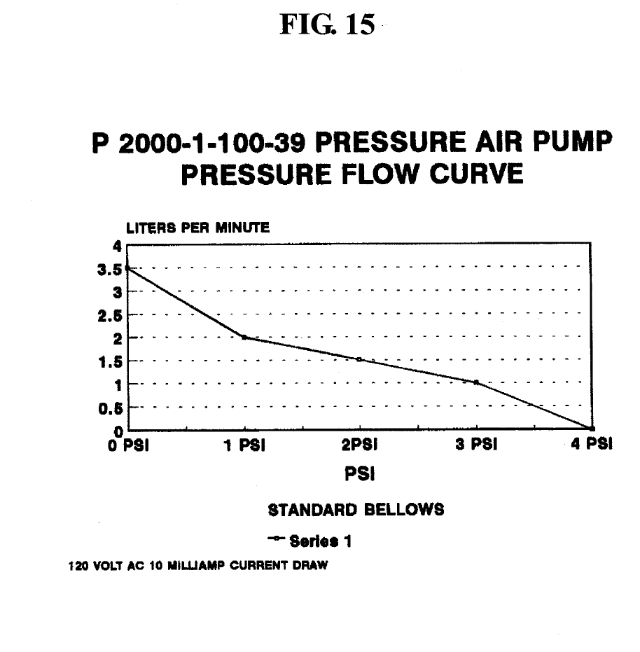

A preferred air pump 110 is made by Electro-Mechanical Mfg. in Ohio. As will be seen in the chart in FIG. 15, the volume of air depends upon the typical depth of the ozone diffuser, the outlet port for the ozone as it enters the water being treated. It is preferable to have the diffuser about 3 meters below the water level so that the ozone and rising air bubbles generated by the ozone generator can mix with the water.

The UV lamp is run by a ballast preferably made by Lightwave. The model is EB-105-03, CAT #GPH793-120/240 TUV 36, GPH 793, Input 120 VAC 60 hz 0.80 A, 240 VAC 50 hz 0.38 A and it runs the lamp at about 40-45V, 560 mA and about 22-24 Watts. Other ballasts with higher output may also be used. Other ballasts with higher output run the same lamp at 850 mA, 33 volts, 28 watts.

The inside of the muffler is cooled by a small 24 Volt muffin fan 130 and the little transformer 132 steps down the voltage from 120 v or 220 v AC for proper operation.

One improvement of this ozone generator is the fact that a very small volume of air enters the elliptical cavity. Only the air drawn by the air pump gets inside. For this reason, it is very easy to filter the air available to the air pump. And, the most important factor, is that the air filter medium can remove over 99% of airborne particles. As stated above, a preferable filter media can be that used for industrial breathing masks used by workers to spray paint or work in chemical and dusty environments. Another preferable filter medium to use is HEPA filter media. HEPA filters can remove at least 99.97% of particles 0.3 (μm) in diameter. Particles of this size are the most difficult to filter and are thus considered the most penetrating particle size (MPPS). Particles that are larger or smaller are filtered with even higher efficiency.

HEPA filters are composed of a mat of randomly arranged fibres. Key metrics affecting function are fibre density and diameter, and filter thickness. The air space between HEPA filter fibres is much greater than 0.3 μm. HEPA filters are designed to target much smaller pollutants and particles are mainly trapped (they stick to a fibre) by one of the following three mechanisms:
1. Interception, where particles following a line of flow in the air stream come within one radius of a fibre and adhere to it.
2. Impaction, where larger particles are unable to avoid fibres by following the curving contours of the air stream and are forced to embed in one of them directly; this increases with diminishing fibre separation and higher air flow velocity.
3. Diffusion, an enhancing mechanism is a result of the collision with gas molecules by the smallest particles, especially those below 0.1 μm in diameter, which are thereby impeded and delayed in their path through the filter and raises the probability that a particle will be stopped by either of the two mechanisms above; it becomes dominant at lower air flow velocities.

Diffusion predominates below the 0.1 μm diameter particle size. Impaction and interception predominate above 0.4 μm. In between, near the 0.3 μm MPPS, diffusion and interception predominate. The initial filter air flow resistance and final filter air flow resistance are typically measured as pressure drop across the filters.

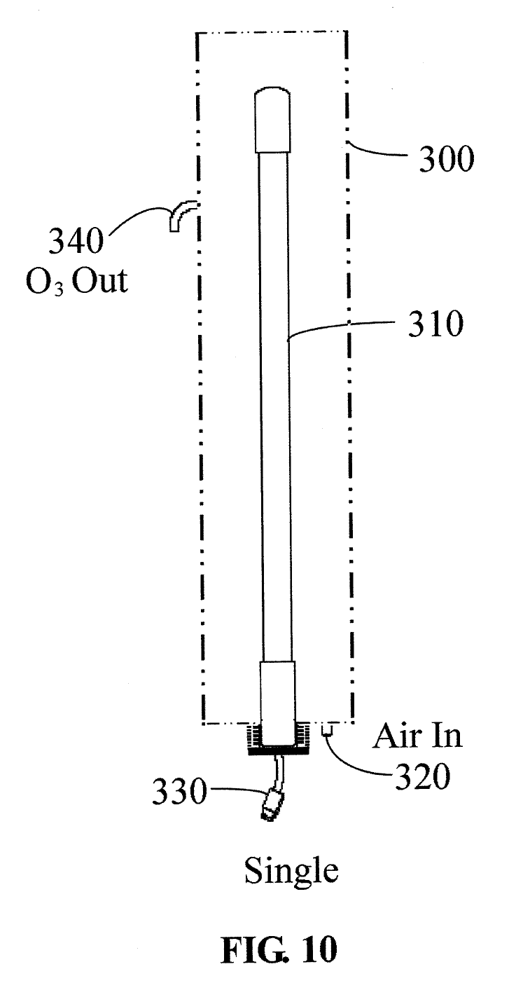

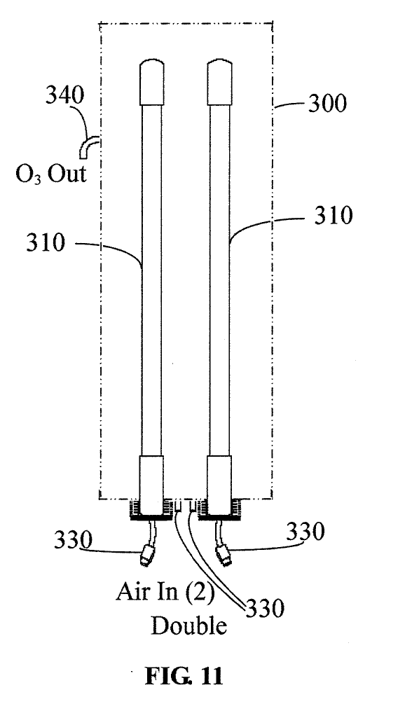

Two embodiments of a UV lamp housing according to the present invention are shown in FIGS. 10 and 11. The lamp housing 300 contains a UV ozone generating lamp 310. Lamp housing 300′ in an alternate embodiment includes two such UV lamps 310. Air from air pump 110 is fed to the lamp housing 300 via an opening 320 in the lamp housing 300. Two such openings 320 may be used for the dual lamp embodiment shown in lamp housing 300′. Power for each UV lamp 310 is provided by cable 330. Ozonated air is output from housing 300/300′ at output port 340. The ozone generator having a single UV lamp preferably has one ballast, one air pump, and one cooling fan. The ozone generator having two UV lamps preferably has two air pumps, two ballasts, and one cooling fan.

In other words, pressurized air enters the lamp housing 300 at the bottom and travels across the inside of the lamp housing and out another stainless steel nipple (340) at the top, which is where another Neoprene or PVC clear tube sends the output ozonated air from the lamp housing 300 to the water tank, pool, or spa.

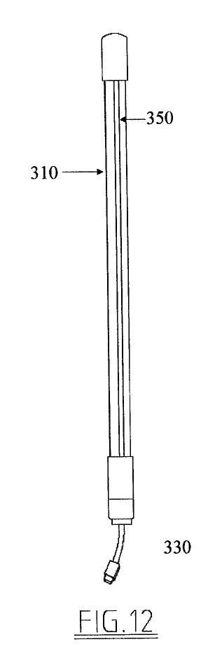

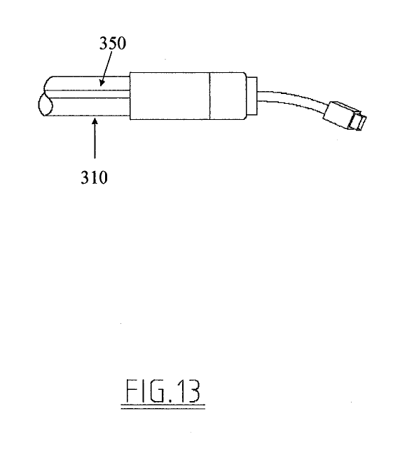

FIG. 12 shows a UV lamp assembly according to the present invention. The UV lamp 310 is wired with the input power provided by cable 330 with a wire 350 connecting the opposite end of the lamp 310 to cable 330. FIG. 13 is a more detailed view of one end of the UV lamp shown in FIG. 12.

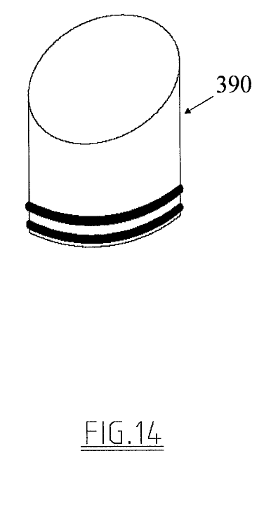

The lamp housing 300 is sealed with o-rings at the “socket” end of the lamp 310 on the bottom where the lamp or lamps are placed inside housing 300 and at the top end preferably by a plastic or glass bulb 390. The ozone lamp housing is preferable sealed with a polycarbonate bulb, shown separately in FIG. 14, that lets the blue light from the ozone producing lamp shine through. A heat resistant glass bulb may also be used. It is an easy and visible signal that the ozone lamp is turned on and the unit is functioning. The bulb shown here has two small o-rings that fit against the stainless steel lamp housing. The o-rings are preferably made of Viton or silicone.

The elliptical case is sealed except for a small inlet 170 to allow ambient air to enter the case. The single UV lamp ozone generator in one embodiment has a 12 mm round air inlet and the double UV lamp ozone generator in another embodiment has a 15 mm round inlet. Both units preferably include an air filter on the inside of the case attached to the air inlet.

The benefits of the new design over previous UV ozone producing generators:
1) The case muffles the sound of the air pump, which is quite noisy especially if you are sitting near by;
2) The case keeps the internal parts (especially the air pump bellows) free of dust, dirt, and insects. The case also reduces relative humidity inside. The internal components can operate more efficiently and longer. For example, the air pump bellows and seals are protected from being contaminated by dust and dirt;
3) The sealed elliptical case reduces the relative humidity inside and reduces corrosion and rust allowing the components to operate longer;
4) The elliptical case pre-treats and pre-micro filters the air sent to the UV lamp assembly. The internal temperature of the elliptical case may reach as high as 55° C. (although in cold outside air, the temperature may be quite low) and, depending on the outside ambient temperature, this heat dehydrates and cooks pathogens and algae spores that might otherwise pass through the air filter;
5) The UV lamp housing has a temperature of about 75° C. and effectively kills or destroys pathogens and spores that reach this chamber, thereby producing high volumes of clean treated air along with the ozone that is pumped from the generator;
6) The ozone and air output from the generator is relatively clean and sterile and relatively free of organic matter, which although lifeless, would still provide nutrients for germs and algae living in the pool or spa or water storage tank used for drinking water or for producing bottled drinking water. When compared to the large quantity of germs and organic matter typically “fed” to a pool or spa or drinking water storage tank by ozone generators using prior art, the new design presented here is a quantum leap forward in the quest to achieve a clean and clear pool or spa without the use of chemicals, salts, or other additives; and,
7) The ozone and air output from the generator allows for the operation of 24 hour use for pools, spas, and water storage tanks used for drinking water because of the low draw of electricity and the low volume of ozone produced. As the air and ozone bubble stream are fed by a diffuser to the storage tank and used in conjunction with a filter, the filtration caused by the air and ozone (water is sucked through the filter due to the rising bubbles) makes water that is very clean. Such 24 hour continuous operation keeps the water cleaner and prevents the growth of bacteria, germs, fungus, algae, and other contaminants in the first place by maintaining an environment that is hostile to pathogens and other contaminants.

Some UV or CD generators might use O₂ feed systems which would also produce clean treated air, but that is a very costly alternative requiring oxygen tanks and automatic feed systems. Other UV or CD generators may use dryers to remove moisture and treat the air before being used, but this also may add considerable expense. Most CD generators do not run 24 hours a day because they produce too much ozone and the water would be too concentrated with ozone. For this reason, they are cycled on and off. However, when the CD generator is turned off, about one hour later there is no more ozone in the water and bacteria and other contaminants can start to grow. For this reason, many ozone systems are used with other chemicals to provide a bacterial kill solution during the times that the ozone generator is turned off.

The double lamp ozone generator is preferably used with pools, spas, and storage tanks used for drinking water (for some storage tank applications, a single lamp ozone generator might be all that is required). The double lamp generator can operate continuously 24 hours a day and, in that time period, it will produce about 6-10 grams of ozone. It also produces about 10,000 liters of clean treated air along with the ozone in the same 24 hour period.

Ozone is produced by pumping filtered (and, in one embodiment, heated) ambient air past a special ozone producing UV lamp housed in a sealed chamber. In a double lamp and double air pump ozone generator, treated air is pumped through the ozone lamp housing at the rate of about 7 liters per minute at an average pressure of 3 psi, depending on the depth of the ozone diffuser in the water. Ozone makes up only about 1% of the total output—the other 99% of the gas stream is air which is relatively free of pathogens, germs, algae spores, dust, organic matter and other impurities. This mixture of air and ozone gas is pumped to an ozone diffuser which is inside a filter or in some cases, a vertical tube filled with water. The diffuser releases thousands of tiny bubbles which cause the water to rise and causes a flow of water through a filter. This combination of an ozone generator and water filtration comprises the basic system for use in the various pools, spas, and water storage tank systems and drinking water factories.

The ozone generator according to the present invention produces a constant flow of ozone and clean air that is useful in many applications. The design restricts the volume of air and filters it before the air goes into a sealed chamber (our muffler). The air entering the chamber is limited in volume to what the air pumps suck in and pump out. As a result of the low volume of air in a sealed chamber, the air in the chamber is preferably heated. This causes the air in the chamber to have a lower relative humidity compared to the ambient air which can have high humidity such as the tropics or some areas of Asia, for example. The air pumped into the UV lamp housing is further heated with the effect that airborne pathogens, pollen, and spores are inactivated. Another benefit is that the generator is quiet and the components last longer because they are kept clean and protected from moisture. Another benefit of the ozone generator according to the present invention is that it can operate 24 hours a day and produces a low volume of ozone and a large volume of clean air and therefore does not have to be cycled on and off like many CD or higher output UV generators. This small “metered” amount of ozone and huge quantity of clean air used to operate an ozone/aeration system is one of the key reasons it can keep pools, spas, storage tanks, and drinking water treatment systems to clean and free of contaminants. Finally, a small muffin fan on the inside of the chamber circulates the air and effectively uses the chamber itself as a heat exchanger. This stirring of the air also helps to keep the air pump and electrical components from getting too hot.

What is claimed is:

1. An ozone generator comprising:
a first sealed container having a first opening for enabling air to enter said first sealed container and a second opening for enabling air to exit said first sealed container;
an air filter positioned at said first opening of said first sealed container for filtering air entering said container;
an air pump positioned inside said first sealed container for drawing air into said container through said air filter and for causing said air to exit through said second opening;
a second sealed container having first and second openings, said first opening operatively connected to said second opening of said first sealed container for receiving into said sealed second container air from said first sealed container, said second opening for enabling air to exit said second sealed container; and
a UV light source positioned inside said second sealed container in a manner so as to cause the air from said first opening in said second sealed container to be radiated by said UV light source as the air flows towards said second opening of said second sealed container, to cause at least some of the air to be converted into ozone gas.

2. The ozone generator of claim 1, further comprising a muffin fan positioned inside the first container to circulate air in said first container.

3. The ozone generator of claim 2, wherein the walls and base of said first sealed container act as a heat exchanger to control the air temperature inside said container.

4. The ozone generator of claim 1, wherein said air pump is a twin bellows type including first and second bellows powered by a drive motor.

5. The ozone generator of claim 1, further comprising an ozone indicator light visible on the exterior of second container.

6. The ozone generator of claim 5, wherein said ozone indicator light comprises a translucent cap on said second container for enabling the visible light being generated by the UV light source to be visible through said translucent cap.

7. The ozone generator of claim 6, wherein said translucent cap comprises a heat resistant plastic.

8. The ozone generator of claim 1, further comprising at least one cooling tube positioned outside of said second container for cooling the air exiting from said second opening of said second container.

9. The ozone generator of claim 1, further comprising a ballast circuit for powering said UV light source, said ballast circuit positioned inside said first sealed container.

10. The ozone generator of claim 1 wherein said air pump has a flow rate of at least 3 liters of air per minute.

11. The ozone generator of claim 1, wherein said air filter filters out particles greater than 10 microns in size.

12. The ozone generator of claim 11, wherein said air filter includes HEPA filter media.

13. The ozone generator of claim 1, further comprising a tube for coupling air flowing out of said second opening in said second container to an ozone diffuser positioned in a reservoir of water.

14. An ozone generator comprising:
a first sealed container having a first opening for enabling air to enter said first sealed container and a plurality of output openings for enabling air to exit said first sealed container;
an air filter positioned at said first opening of said first sealed container for filtering air entering said container;
a plurality of air pumps positioned inside said first sealed container for drawing air into said container through said air filter, each air pump output coupled to a separate said output opening for causing said air to exit through said output openings;
a second sealed container having a plurality of input openings and one second opening, said input openings operatively connected to corresponding said output opening of said first sealed container for receiving into said sealed second container air from said first sealed container, each input opening coupled to a separate cavity in said second container, said second opening for enabling air to exit said second sealed container; and
a plurality of UV light sources each positioned inside a respective cavity in said second sealed container in a manner so as to cause the air from said input openings in said second sealed container to be radiated by said UV light sources as the air flows through to said second opening of said second sealed container, to cause at least some of the air to be converted into ozone gas.

15. The ozone generator of claim 14, wherein each said air pump has a flow rate of at least 3 liters of air per minute.
